(19) United States
(12) Patent Application Publication
(10) Pub. No.: US 2022/0386912 A1
(43) Pub. Date: Dec. 8, 2022

(54) Blood collection device and method for the self-collection of blood by a user

(71) Applicant: STICHTING HET NEDERLANDS KANKER INSTITUUT-ANTONI VAN LEEUWENHOEK ZIEKENHUIS, CX AMSTERDAM (NL)

(72) Inventors: Nicas VAN DEN BRINK, CX AMSTERDAM (NL); Huub Harmen VAN ROSSUM, CX AMSTERDAM (NL)

(21) Appl. No.: 17/775,164

(22) PCT Filed: Nov. 5, 2020
(86) PCT No.: PCT/NL2020/050691

§ 371 Date: May 6, 2022

(30) Foreign Application Priority Data
Nov. 7, 2019 (NL) 2024178

(51) Int. Cl. A61B 5/15

(52) U.S. Cl. CPC A61B 5/150022; A61B 5/150305; A61B 5/150343; A61B 5/150267

(57) ABSTRACT

A blood collection device for the self-collection of a blood sample by a user, wherein,
a base element has a width of more than 1.75 in and including an upstanding wall which defines a receive opening, the upstanding wall having a height of less than 1.75 in;
a collection container is configured for placement in said receive opening in a substantially upright orientation;
the finger portion comprising a blood collection hole that provides a fluid channel between the collection container and the finger portion;
wherein at least the base element is a separate part, configured to be shipped in a de-assembled state with respect to the other parts of the blood collection device,
wherein the blood collection device is configured to be assembled by a user after receiving said blood collection device.

### CROSS-REFERENCE TO RELATED APPLICATIONS

This application is US National Stage of International Patent Application PCT/NL2020/050691, filed Nov. 5, 2020, which claims benefit of priority from Netherlands Patent Application NL2024178, filed Nov. 7, 2019, the contents of both of which are incorporated herein by reference.

### BACKGROUND

The present invention relates to a blood collection device.

Such devices are known in general and can typically be categorised into two categories. On the one hand, there are devices which allow the self-collection of a blood sample by a user, i.e. the collection of a blood sample by a user without any help of a second person such as a physician. These devices are however usually rather complex, and therefore relatively costly. Another disadvantage of such devices is that they are typically relatively large, which makes it impossible to send these devices to users via letterbox post. Instead, it has to be send as a package (which of course results in higher cost). US 2015/0351676 A1 e.g. describes such a device.

On the other hand there are devices which are smaller, but these typically cannot be used by a user for the self-collection of blood. That is, these types of devices require a second person, such as a physician, to successfully collect a blood sample. With such devices, typically three hands (and, thus, at least two persons) are needed. Firstly there is the hand comprising the finger of the user from which the blood sample is to be taken. Secondly, there is a hand which is needed to “push out” the blood from the finger of the user. And thirdly there is a hand which is needed to hold the blood collection device and collect the blood sample. The requirement of three hands makes it rather impossible for users to collect blood themselves with such devices.

### SUMMARY OF THE INVENTION

Hence, there is a need for a device which combines the good aspects of both these types of devices, i.e. there is a need for a device which allows a user to collect a blood sample himself (herself), and which is sufficiently compact to allow it to be send via lettermail post.

It is an object of the present invention to provide such a device.

Therefore, a blood collection device for the self-collection of a blood sample by a user is provided, the blood collection device comprising a base element, a collection container, and a finger portion,

the base element having a width of more than 1.75 in and including an upstanding wall which defines a receive opening for receiving the collection container, the upstanding wall having a height of less than 1.75 in;

the collection container being configured for placement in the receive opening of the base element in a substantially upright orientation;

the finger portion being configured for placement on top of the collection container and comprising a blood collection hole which, in an assembled state of the blood collection device, enables a flow of blood from the finger portion into the collection container,

wherein at least the base element is a separate part, configured to be shipped to a user in a de-assembled state with respect to the other parts of the blood collection device, and

wherein the blood collection device is configured to be assembled by a user after receiving said blood collection device and before using said blood collection device.

The blood collection device according to the invention advantageously is a very simple device, having only a few components, and having low production cost. The device is very simple to assemble for a user, and may advantageously be shipped to said user via letterbox post, such that shipping costs are minimal. In use of the blood collection device advantageously a “third hand” for the collection of a blood sample is not needed so that the device may truly be used for the “self-collection” of a blood sample by the user himself (herself).

The device according to the invention comprises a base element, which base element is a separate part compared to the other components of the blood collection device in the sense that it can be de-assembled and assembled together with the other components of the blood collection device to form the blood collection device in an assembled state. With reference to the below text where this is explained in more detail, it is noted that the other components of the blood collection device may also be de-assembled with respect to each other, but this is not required. Some or all of the other components may be integrated with each other.

The base element is configured to be shipped to a user in a de-assembled state with respect to the other parts, such that a small and comprehensive package results in the de-assembled state of the blood collection device, a package which may be send through lettermail post (which according to Dutch regulations in force at the filing date of the present application means at least that the height of the package should be less than 32 mm). In other countries other requirements may however apply for lettermail post and the design of the blood collection device may easily be adapted to such requirements without departing from the inventive concept underlying the invention. Purely as an example and to the best knowledge of the applicant, the European standard for letter boxes, EN 13724:2002, as in force on the date of filing, mentions an aperture height of 30-35 mm; Canadian law mentions a height of 40 mm on the filing date; USA law mentions a height of 1.75 inch on the filing date; and Australian law mentions a height of 30 mm on the filing date.

The base element is further configured to be assembled with the other parts of the blood collection device by a user after said user has received the blood collection device via lettermail post and before using said blood collection device. As is explained in more detail below, the design of the blood collection device is advantageously very simple such that any user is able to assemble the blood collection device without difficulties and without the risk of a misassembly.

Alternatively phrased, the blood collection device can be de-assembled (it is however by no means required that the device has been assembled before being de-assembled), e.g. to ship it to a user in a de-assembled state, and the blood collection device can be assembled, e.g. to use it for collecting a blood sample in an assembled state.

It has been an important insight of the inventors that a base element having a width of more than 1.75 in ensures a stable support surface when the blood collection device is used, and that the possibility to de-assemble it with respect to the other components of the blood collection device makes it possible to send the base element via lettermail post at the same time. In other words, by providing a blood collection device that can be de-assembled, the two functions of providing a stable support and being sufficiently compact to allow shipping via letterbox post can be fulfilled at the same time. Compared to the solutions of the prior art, the stable support by the base element ensures that no “third” hand of a user (or a second person) is needed to hold the blood collection device. Instead, the blood collection device is self-supporting. This makes the collection of blood with just two hands, i.e. by the user him/herself possible.

The base element should then have a height of less than 1.75 in to allow it to be send via lettermail post in the de-assembled state. As the width is more than 1.75 in, it is not possible to send the blood collection device via lettermail box in its assembled state and therefore, advantageously, it is possible to de-assemble the base element with respect to the other elements of the blood collection device.

In the assembled state of the blood collection device, the base element may form a stable foot which may be placed by a user on a support surface such as a desk, a table, or another relatively large, substantially horizontal surface. The collection container is placed inside the base element, in the receive opening thereof, and extends upwards with respect to the base element.

In use, a user of the blood collection device may typically assemble the blood collection device, prick his/her finger with a lancet, wait and/or work the finger until a drop of blood is visible, place said finger on the finger portion of the blood collection device, and wait until enough blood is drawn from the finger of the user into the collection container through the fluid channel. The upright positioning of the collection container ensures that blood drawn from the finger of the user is transported towards the collection container along with the force of gravity, such that actively pushing blood out of the finger of the user may be needed to a lesser degree or not at all, and hence allowing the “self-collection” of a blood sample.

For example, the user may prick the ring finger or the middle finger with the lancet, e.g. in a top section thereof.

Advantageously, the blood collection device does not comprise any moving parts, such that both assembly and manufacturing are fast and cheap.

The finger portion comprises a blood collection channel including a blood collection hole, a width of the blood collection channel decreasing in a direction from an upper surface of the finger portion to the collection container. At the bottom surface of the finger portion, the blood collection hole extends into a blood collection channel, the channel extending towards the collection container in the assembled state of the blood collection device. A cross-section of said channel is larger near the top thereof (i.e. near the finger portion) than near the bottom thereof (i.e. near the collection container). For example, the cross-sectional shape of the channel may be tapered or conical. It was surprisingly found by the applicant that such a shape allows any air present in the collection container after assembly to leave the collection container, allowing the blood to more easily flow through the blood collection hole (channel), the fluid channel, and into the collection container. An almost immediate flow is observed once the user places his/her (pricked) finger on the finger portion. The specified shape of the blood collection channel furthermore helps to efficiently direct blood towards and into the collection container. The specified shape also helps to direct blood towards and into the collection container in a clean way, i.e. with less sloppiness.

In other words, a blood collection channel having a width which decreases in a direction from an upper surface of the finger portion to the collection container provides an air outlet channel for any excess air in the collection container after assembly.

In an embodiment, the blood collection device further comprises a capillary tube that, in an assembled state, is arranged between the blood collection hole and the collection container, the capillary tube defining said fluid channel. When a capillary tube is provided, it becomes very easy to collect relatively small blood samples.

In an embodiment, the capillary tube extends into the collection container, the collection container having a larger width (diameter) than the capillary tube.

In an embodiment, an outer width of the capillary tube is larger than a width of the blood collection hole. After assembly, air may be present in the collection tube and/or the internal space of the finger portion. This air may create a relative overpressure which prevents blood deposited on the finger portion by the user to easily flow into the collection container. When the outer width of the capillary tube is larger than a width of the blood collection hole, this prevents the capillary tube to fill and obstruct the blood collection hole/blood collection channel. This in turn allows any air in the collection container/internal space of the blood collection device to leave through the blood collection hole, so that an overpressure in the collection container/internal space of the finger portion is reduced and a more easy flow of blood into the collection container may be obtained.

In other words, an outer width of the capillary tube being larger than a width of the blood collection hole provides an air outlet channel for excess air in the collection container after assembly.

In an embodiment a side wall of the finger portion comprises an air outlet hole. Said air outlet hole may act as an air vent to allow any air residing in the collection container and/or the internals of the blood collection device (that creates an overpressure) to escape therefrom, so that a more easy flow of blood from the finger portion into the collection container may be obtained. In other words, the air outlet hole provides an air outlet channel for excess air in the blood collection device after assembly. Advantageously, when said air outlet hole is provided in the side wall of the finger portion, it cannot be obstructed by blood and is always free.

In an embodiment the blood collection device further comprises an insert member configured to be inserted into the finger portion and for receiving the capillary tube, to fixate the capillary tube with respect to the finger portion. The insert member may be made of a different material than the finger portion. For example, the finger portion may be made form a relatively stiff material, and the insert member may e.g. be made from a relatively flexible material. This allows to pressure-fit the insert member with respect to the finger portion, and to pressure-fit the capillary tube with respect to the insert member, and thus to secure the capillary tube with respect to the finger portion. For example the insert member may be pressure-fit in a hollow side wall (or a hollow internal wall) of the finger portion. For example, the capillary tube may be pressure-fit in a capillary tube receiving channel of the insert member. The insert member may have recesses in its outer circumferential surface, which recesses may define air outlet channels for excess air in the collection container after assembly.

In this embodiment, preferably the capillary tube receiving channel of the insert member has a non-circular, e.g. square, cross-section, and the capillary tube has a circular cross-section. This allows on the one hand to pressure-fit the capillary tube in the insert member, e.g. when the outer diameter of the capillary tube is approximately the same as an inner width of the capillary tube receiving channel. This allows on the other hand that an outflow channel for any air in the collection container is provided between the outer side of the capillary tube and the capillary tube receiving channel. The open space between the outer side of the capillary tube and the capillary tube receiving channel defines an air outlet channel.

Alternatively, a capillary tube receiving channel of the insert member may have a circular cross-section while the capillary tube has a non-circular cross section, and wherein the sizes of the capillary tube and the capillary tube receiving channel are matched to each other to allow a press-fit of the capillary tube in the capillary tube receiving channel.

Preferably, when the blood collection device comprises an insert member, the finger portion, the insert member and the capillary tube are pre-assembled in the de-assembled state. Alternatively, when the blood collection device does not comprise an insert member, the finger portion and the capillary tube are pre-assembled in the de-assembled state. As explained in the above, it may be desirable that any air residing in the collection container after assembly can leave the collection container. Therefore, flow channels for excess air (i.e. air outlet channels) are preferably provided, where several different embodiments of such flow channels are described in the above. One or more of such flow channels for air may be present in the blood collection device. To ensure that the flow channels are not closed as a result of incorrect assembly, preferably the capillary tube is pre-assembled with respect to the finger portion in the de-assembled state. Accordingly, in the de-assembled state not all components of the blood collection device need to be separated from each other. Some of the components may be pre-assembled in the de-assembled state.

In an embodiment an upper surface of the finger portion has an elongate shape. Advantageously an elongate shape makes it easy and intuitive for a user to place his/her finger on the finger portion, as a finger also has an elongate shape. The finger portion may e.g. have an elliptical, rounded shape. The finger portion may e.g. have an upper surface which is slanted towards the blood collection hole/blood collection channel. This matches the natural shape of a finger, and transports any blood automatically towards the blood collection hole. The finger portion may have an upstanding wall portion. The finger portion may have a “lip” where a root of the finger is to be placed, whereas the blood collection hole is arranged at the opposite side of the lip. All these factors, individually and combined, may make it easy and intuitive for a user to place a finger of the user on the finger portion in the most optimal way, and to collect the blood sample in the most efficient way, offering a pleasant experience for the user. To provide a stable and secure structure for the placement of a finger, a length of the finger portion may be more than 1.75 in. In that case, the finger portion is preferably send in a de-assembled state with respect to the collection tube and preferably also with respect to the base element. Preferably a height of the finger portion is less than 1.75 in, to allow it to be shipped via lettermail post in a de-assembled state.

Advantageously, the elongate shape of the finger portion, and the placement of the blood collection hole, ensures that a user intuitively knows how to place the (pricked) finger on the finger portion for the sampling of blood.

In an embodiment, a width of the base element is larger than 2.0 in. In general, a wider base element will result in a more stable blood collection device. Preferably, a circumferential shape of the base element is circular, but other shapes are well conceivable. Preferably the width of the base element exceeds the length of the finger portion.

In an embodiment, the base element comprises a receive pocket including the receive opening for receiving the collection container in the assembled state of the blood collection device. The receive pocket may e.g. be formed by an internal wall of the base element. A receive pocket ensures a stable and secure assembly of the collection container with respect to the base element, as the contact area between the base element and the collection container is increased compared to when e.g. only a receive opening is defined in the base element for receiving the collection container.

In an embodiment the collection container comprises at least one barline for indicating a fill level of the collection container. Preferably, the collection container is transparent. The barline may indicate the level till which the container is to be filled, e.g. 50 μl, 100 μl or 200 μl. For different tests, different amounts of blood may be desired. Therefore the collection container may comprises several such barlines, each indicating a different fill level. Instructions may then be provided along with the blood collection device regarding the amount of blood that is to be collected. To also see whether the amount of blood collected is above or at the required fill level, the collection container is preferable transparent, although other ways to indicate this are known to a person skilled in the art.

In an embodiment the base element and/or the finger portion are manufactured via an injection moulding process, the base element and the finger portion preferable being made from polypropylene. An injection moulding process typically requires relatively expensive moulds and thus a relatively large initial investment, but has a relatively low price per piece such that an injection moulding process may be cost-effective when a large amount of devices, e.g. more than 10,000, is produced.

In an alternative embodiment the base element and/or the finger portion are manufactured via an 3D printing process, preferably via a selective laser sintering process, the base element and the finger portion preferable being made from PA2200. A 3D printing process has a relatively low initial investment (only the 3D-printer, which is cheap compared to an injection mould), but has a relatively high price per piece such that a 3D printing process may only be cost-effective when a relatively low amount of blood collection devices is made, e.g. less than 1,000. For customization purposes, it may however be desired to have small batches such that 3D printing a desirable. This e.g. allows branding to be provided on the side of the blood collection device. A particular advantage of PA2200 is that it is a biocompatible plastic.

A second aspect of the present invention relates to a base element for a blood collection device, the base element having a width of more than 1.75 in and including an upstanding wall which defines a receive opening for receiving a collection container or a blood collection device, the upstanding wall having a height of less than 1.75 in,

wherein the base element is a separate part, configured to be shipped to a user in a de-assembled state with respect to the other parts of the blood collection device or with respect to the blood collection device, and

wherein the base element is configured to provide a stable support for the blood collection device in an assembled state.

Making use of the same inventive concept as described in relation to the first aspect of the invention, any known blood collection device may be provided together with a separate base element that is configured to provide a stable support. Any blood collection device can then be placed on a support and be used by a user for the self-collection of a blood sample without the need of a “third hand” or second person. The base element of this second aspect may be provided “in addition to” a blood collection device, or may be provided “as a part of” a blood collection device.

A third aspect of the present invention relates to a blood collection kit comprising the blood collection device as detailed in the above, a lancet, and a cap for sealing the collection container. Preferably the blood collection kit is packed in a sterilized bag. Optionally the blood collection kit also comprises a return envelope and/or an analysis tool for self-analysis of the collected blood sample.

A fourth aspect of the present invention relates to a method for collecting a blood sample, comprising the steps of:

shipping a blood collection device as detailed in the above or a blood collection kit as detailed in the above to a user by letterbox post, a base element of the blood collection device being de-assembled with respect to the other parts of the blood collection device during shipment,

assembling the blood collection device;

pricking a finger of a patient to initiate a blood flow;

placing the finger of the patient on the finger portion of the blood collection device;

collecting the blood of the patient in the collection container;

de-assembling the blood collection device;

returning the collection container, inclusive of the blood collected therein;

wherein the steps of assembling the blood collection device, pricking the finger of a patient, placing the finger of the patient on the finger portion, collecting the blood, and de-assembling the blood collection device are all carried out by the patient him/herself.

Optionally, the finger of the user may be prepared before the step of pricking the finger. For example, the finger may be held under a running flow of hot water to enhance blood flow.

A fifth aspect of the present invention relates to a blood collection device for the self-collection of a blood sample by a user, the blood collection device comprising a base element, a collection container, and a finger portion,

the base element including a hinge member for moving the base element between a folded state and an unfolded state (and vice versa) and an upstanding wall which defines a receive opening for receiving the collection container, the base element, in the unfolded state, having a width of more than 1.75 in and, in the folded state, having a height of less than 1.75 in;

the collection container being configured for placement in the receive opening of the base element in a substantially upright orientation;

the finger portion being configured for placement on top of the collection container and comprising a blood collection hole which, in an assembled state of the blood collection device, provides a fluid channel between the collection container and the finger portion,

wherein the base element is configured to be shipped to a user in the folded state, and

wherein the base element is configured to be unfolded by a user after receiving said blood collection device and before using said blood collection device.

The advantages described in the above in relation to the first aspect of the present invention are equally applicable to the second, third, fourth and fifth aspect of the present invention.

Specific embodiments described in relation to the first aspect are equally applicable to the second, third, fourth and fifth aspect of the present invention.

### BRIEF DESCRIPTION OF THE FIGURES

These and other aspects of the invention are elucidated further with respect to the below figures, wherein like parts are indicated with the same reference numerals. Herein,

FIGS. 1A and 1B schematically illustrate a side view of an embodiment of the blood collection device according to the present invention in both its assembled and its de-assembled state;

FIGS. 2A-2D schematically illustrate isometric views of various embodiments of the blood collection device according to the present invention;

FIG. 3 schematically illustrates a partial longitudinal cross-sectional view of an embodiment of the blood collection device according to the present invention;

FIG. 4 schematically illustrates a top view of an assembly of an insert member and a capillary tube;

FIG. 5 schematically illustrates an exploded view and an assembled state of an embodiment of a blood collection device according to the present invention;

FIG. 6 schematically illustrates an embodiment of a blood collection kit according to the second aspect of the invention;

FIGS. 7A and 7B schematically illustrate a longitudinal cross-sectional view of an embodiment of the blood collection device according to the invention; and

FIGS. 8A and 8B schematically illustrate an embodiment of a blood collection device according to the invention, wherein a base element thereof is foldable.

### DETAILED DESCRIPTION OF THE FIGURES

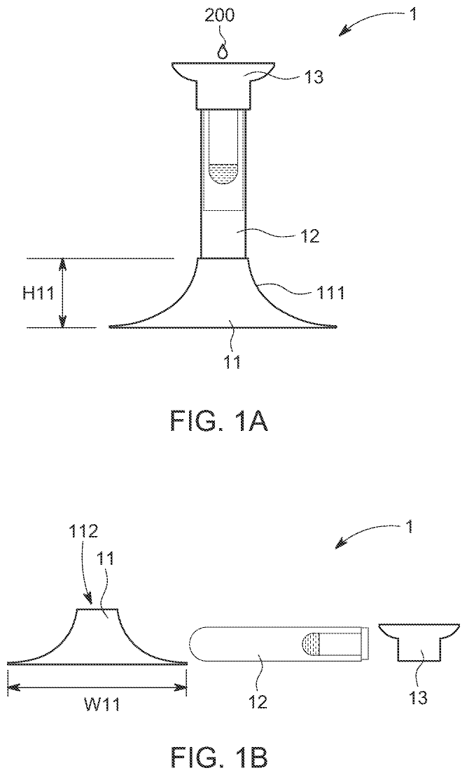

With reference to FIGS. 1A and 1B the blood collection device 1 generally comprises three components: a base element 11, a collection container 12 and a finger portion 13. Each of the different components will be described individually in more detail below. Importantly, at least the base element 11 is a separate part compared to the other components 12, 13. Also the collection container 12 and the finger portion 13 are here however separate parts compared to each other and compared to the base element 11. As the three components 11, 12, 13 are all separate parts, they can be assembled and de-assembled with respect to each other. This allows to ship the blood collection device 1 to be shipped to a user in a de-assembled state. As is visible in FIG. 1B, in the de-assembled state the blood collection device 1 advantageously fits inside a box that has a height of 1.75 in, a length of 15 in (not indicated) and a length of 10 in (not indicated). This allows the blood collection device to be send to a user via letterbox post. The inventive concept underlying the invention can of course easily be adapted to other national postal service requirements without departing from the scope of the invention). Due to the simplicity of the blood collection device 1, a user can very easily assemble the blood collection device 1 after having received it and before using it to collect a sample of blood 200.

The collection container 12 may be a standardized part and is preferably a “buy-in” piece. The advantage of using a standardized collection container is that it may be accepted for testing purposes by any blood testing laboratory. The finger portion 13 and the base element 11 may in turn e.g. be made via an injection process, or e.g. via 3D printing process.

As is indicated in FIGS. 1A and 1B, the base element 11 of the blood collection device 1 has a height H11. The height is less than 1.75 in, preferably less than 1.1 in e.g. less than 1.0 in such that the base element 11 can be placed in e.g. a cardboard box and the resulting package is still thinner than 1.75 in.

The height H11 of the base element 11 is defined by an upstanding wall 111, which is hollow at the inner side thereof and defines a receive opening 112 for receiving the collection container 12. Preferably the shape of the receive opening 112 is adapted to the shape of the collection container 12 such that the collection container 12 neatly fits inside the receive opening 112. Preferably the collection container 12 and the receive opening 112 are circular.

The base element 11 has a width W11 of more than 1.75 in. This ensures a stable and trust-worthy design when the blood-collection device is assembled. For example, the width W11 of the base element 11 may be larger than 2.0 in.

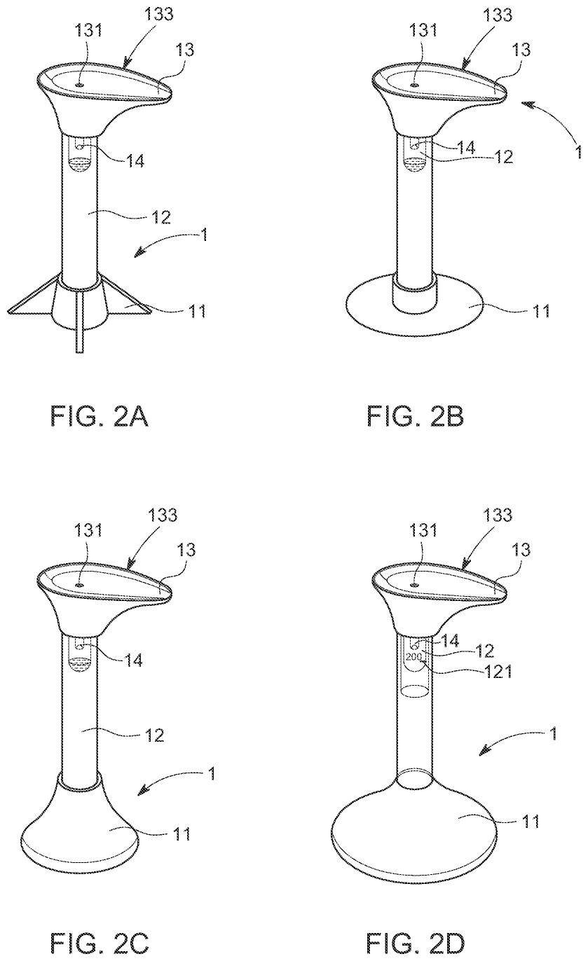

Preferably the width W11 of the base element 11 is less than 10 in, to allow it to fit within the above-mentioned postal dimensions for letterbox mail. With reference to FIGS. 2A-2D, several different shapes of the base element 11 are schematically shown. In FIG. 2A, the base element 11 is cross-shaped, having four legs (of which one is hidden from view). A width of the base element 11 is then defined from one outer end of a leg to the outer end of the opposing leg. Of course, the base element may also have any other number of legs, preferably at least three, such as three, four or five. A skilled person will find it very easy to determine the “width” of a cross-shaped base element 11 with an uneven amount of legs. It is in general at least equal to twice the net length of a leg, plus the diameter of the receive opening.

In FIGS. 2B-2D the shapes of the base elements 11 are comparable. They all have a circular base area, being sloped towards the receive opening in a more or less degree. In FIG. 2B the upstanding wall extends from a base area at an angle of about 90°, whereas said angle is smaller in FIG. 2C and even smaller in FIG. 2D. The width of the base element 11 is smaller in FIG. 2C than in FIG. 2D, with FIG. 2B in the middle. In terms of proportion, it is preferred when the width of the base element 11 is larger than a length of the finger portion 13.

Further visible in the isometric views of FIGS. 2A-2D is the shape of the finger portion 13. The finger portion 13 comprises a blood collection hole 131 and can be placed on top of the collection container 12 in the assembled state of the blood collection device 1. An upper surface 133 of the finger portion 13 here has an elongated shape and is slanted, the lowest point of the upper surface 133 preferably being arranged at or near the blood collection hole 131.

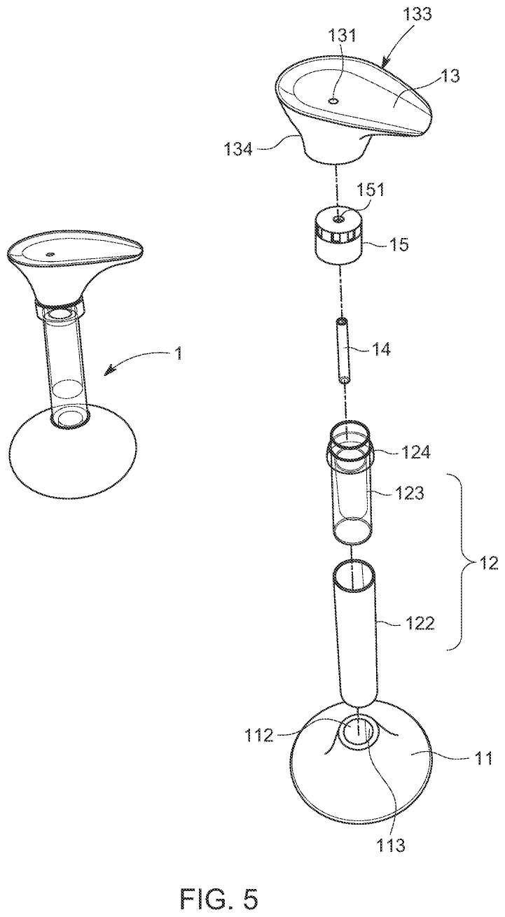

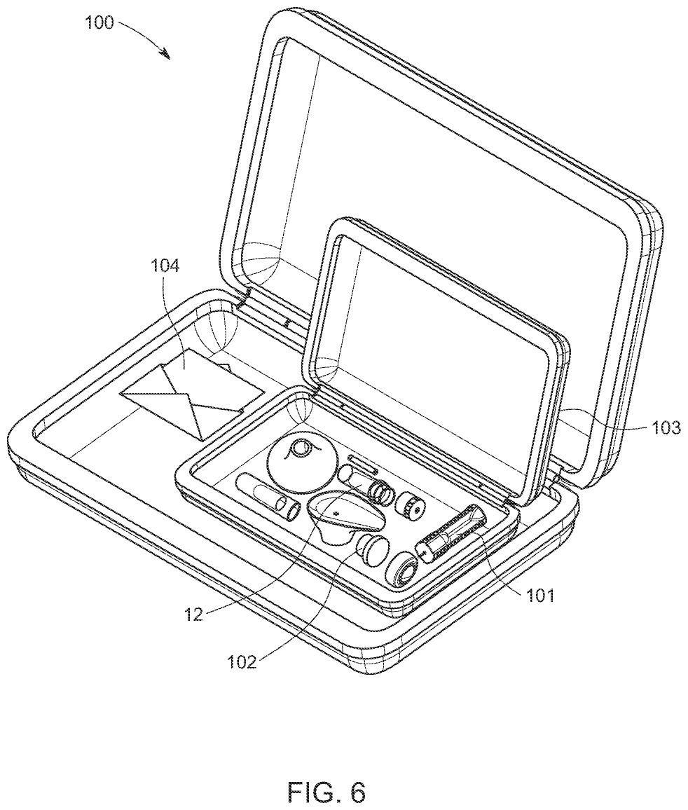

Further visible in the isometric views of FIGS. 2A-2D is a transparent collection container 12. In the shown embodiments, said collection container may comprise an outer tube 122 (see e.g. also FIG. 5) which is placed in the base element 11 and an inner tube 123 (see e.g. also FIG. 5) which is used for the collection of the blood sample. The outer tube 122 may be a standardized part, which is accepted by any test laboratory for testing purposes. Also the inner tube 123 may be a standardized part. The outer tube (and thus the collection container 12) is placed in the receive opening of the base element 11. The outer tube and the inner tube are oriented in an upright position. Again with reference to the exploded view of FIG. 5, the inner tube 123 may e.g. comprise an outer rim 124 that in an assembled state of the blood collection device rests on an upper face of the outer tube 122.

In FIG. 2D a bar line 121 is illustrated. The bar line 121 may e.g. be provided on the outer tube or on the inner tube and indicates a fill level of the collection container 12. In the shown embodiment the “200” may e.g. indicate that when the collection container 12 is filled until bar line 121, 200 μl of blood is collected.

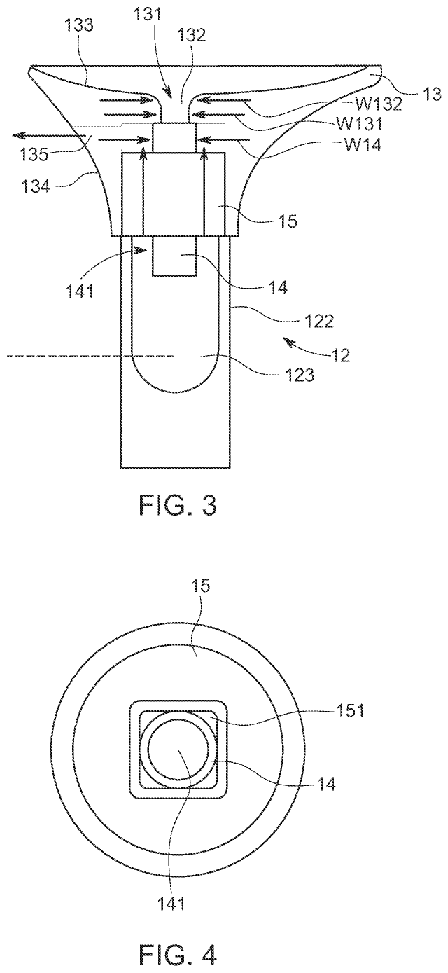

Further visible in FIGS. 2A-2D is the capillary tube 14, which will be discussed in more detail with respect to FIGS. 3 and 4. The capillary tube 14 is preferably smaller in diameter than the collection container 12 and preferably extends into the collection container 12, as is shown here.

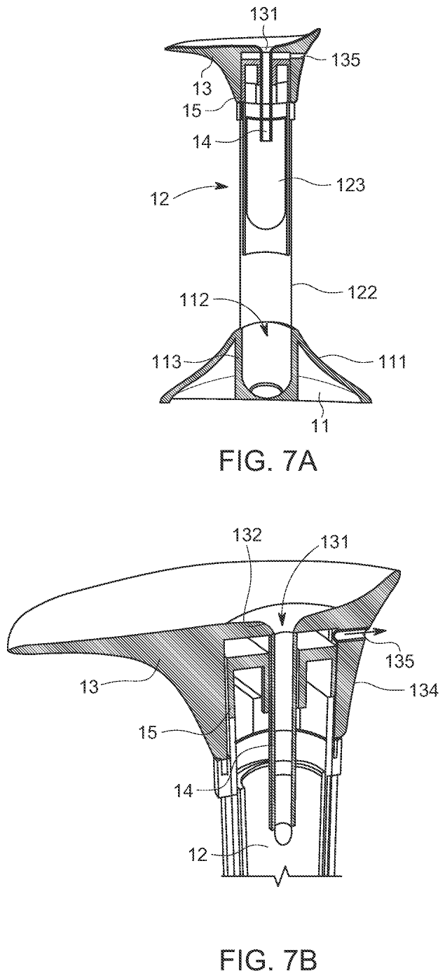

Turning to FIGS. 3, 7A and 7B now, which will be described simultaneously, a longitudinal cross-sectional view of the blood collection device 1 is visible. FIGS. 3 and 7B shows the finger portion 13, the capillary tube 14, the insert member 15, and the collection container 12 with outer tube 122 and inner tube 123 in an assembled state. FIG. 7A shows the entire blood collection device 1. As visible, the upper surface 133 of the finger portion 13 is slanted and directed towards blood collection hole 131. In the shown embodiments the finger portion 13 comprises a blood collection channel 132, formed by a downwardly extending wall and the blood collection hole 131 at the bottom of it. The shape of the blood collection channel 132 is here conical, a width W132 of the channel 132 decreasing in a direction from the upper surface 133 of the finger portion 13 towards the collection container 12.

In the present embodiment, the width of the blood collection channel 132 at the blood collection hole 131, denoted W131 and here the smallest width of the blood collection channel 132, is larger than an outer width W14 of the capillary tube 14. For example, the width W131 of the blood collection hole 131 may be about 3 mm. This difference in size between the capillary tube 14 and the blood collection hole 131 ensures that the capillary tube 14 advantageously abuts the blood collection channel 132 and does not extend up to or beyond the upper surface 133 of the finger portion 13.

Further visible in FIGS. 3, 7A and 7B is an air outlet hole 135. Said air outlet hole 135 may assist any air in the collection container 12 and/or in the inner tube 122 of the collection container 12 to leave said collection container 12 and/or inner tube 122 such that blood may more easily flow from the finger portion 13 into the collection container 12. In other words, any overpressure in the collection container 12, created by excess air therein, may be resolved by having an air outlet 135 in the finger portion 13. The flow of excess air is schematically illustrated by arrows in FIG. 3. The air outlet hole 135 may e.g. have a diameter of between 0.5 mm and 3 mm, such as approximately 1 mm or approximately 2 mm.

As shown, the capillary tube 14 provides a fluid channel 141 for blood between the finger portion 13 and the collection container 12.

As shown, the bottom side of the finger portion 13 is hollow, such that the insert member 15 may be inserted therein.

As is shown in FIG. 7A, the base element 11 may comprise a receive pocket 113 including the receive opening 112 for receiving the collection container 12 in the assembled state of the blood collection device 1.

It is preferred that the finger portion 13, the insert member 15 and the capillary tube 14 are pre-assembled before being shipped to the user.

As is better visible in FIG. 4, said insert member 15 comprises a capillary tube receiving channel 151 for receiving and fixating the capillary tube 14 therein. Via the insert member 15, the capillary tube 14 may thus be fixed with respect to the finger portion 13.

Whereas the cross-section of the capillary tube receiving channel 151 of the insert member 15 is substantially square, the cross section of the capillary tube 15 is substantially circular. As the width of the channel 151 substantially matches the outer diameter of the capillary tube 14, said capillary tube 14 may be press-fit in the capillary tube receiving channel 151.

FIG. 5 again shows a schematic overview of the blood collection device 1, wherein FIG. 5A shows the blood collection device 1 in an assembled state, and FIG. 5B shows an exploded view thereof.

FIG. 6 shows a blood collection kit 100 which comprises the blood collection device 1 as described in the above, a lancet 101, a cap 102 for sealing the collection container 12 after blood is collected therein, and a return envelope 104. Optionally also an analysis tool 105 (not shown) for self-analysis of the collected blood may be part of the blood collection kit 100. Preferably at least the lancet 101, the cap 102 and the components of the blood collection device 1 are packed in a sterilized bag 103.

A blood sample may be collected by a user by:

firstly shipping a blood collection device 1 or a blood collection kit 100 to a user by letterbox post, the blood collection device 1 being in a de-assembled state during shipment;

secondly assembling the blood collection device 1, preferably by the user;

thirdly pricking a finger (schematically illustrated by the drop of blood 200 in FIG. 1) of a patient to initiate a blood flow;, preferably by the user;

fourthly placing the finger 200 of the patient on the finger portion 13 of the blood collection device 1, preferably by the user;

fifthly collecting the blood of the patient in the collection container 12, preferably by the user;

sixthly de-assembling the blood collection device 1, preferably by the user;

seventhly sealing the collection container 12 with a cap 102, preferably by the user; and

finally shipping the collection container 12, inclusive the blood collected therein, to a blood analysis laboratory where the testing can be performed, preferably by the user. In an embodiment the collection container 12, comprising outer tube 122 and inner tube 123 may be send back in its entirety. In another embodiment, only the inner tube 123 may be returned by the user.

Optionally the blood collection kit 100 comprises a self-testing tool, which can be used by the user to perform a test on the collected blood.

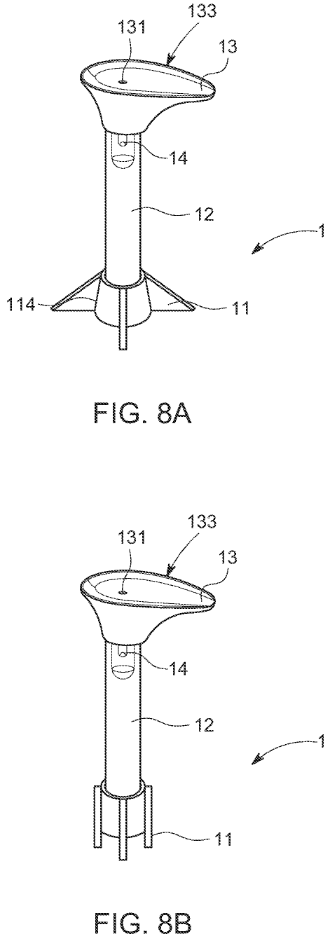

FIG. 8A finally shows an embodiment of a blood collection device 1 which is similar to the embodiment of FIG. 2A. The base element 11 of the blood collection device 1 of FIG. 8A however additionally comprises a hinge member 114 which allows the base element 11 to be moved between a folded state and an unfolded state (and vice versa), such that the base element 11 may be shipped to a user in a compressed, i.e. folded, state and be unfolded by a user after receiving the blood collection device 1.

It is noted that in the shown embodiment the base element 11 may also be de-assembled with respect to the collection container 12, but this is not required when the base element 11 may be compressed by folding in hinge members 114.

Clauses:
1. A blood collection device for the self-collection of a blood sample by a user, the blood collection device comprising a base element, a collection container, and a finger portion,

the base element having a width of more than 1.75 in and including an upstanding wall which defines a receive opening for receiving the collection container, the upstanding wall having a height of less than 1.75 in;

the collection container being configured for placement in the receive opening of the base element in a substantially upright orientation;

the finger portion being configured for placement on top of the collection container and comprising a blood collection hole which, in an assembled state of the blood collection device, enables a flow of blood from the finger portion into the collection container;

wherein at least the base element is a separate part, configured to be shipped to a user in a de-assembled state with respect to the other parts of the blood collection device, and

wherein the blood collection device is configured to be assembled by a user after receiving said blood collection device and before using said blood collection device.
2. The blood collection device according to clause 1, wherein the finger portion comprises a blood collection channel including the blood collection hole, a width of the blood collection channel decreasing in a direction from an upper surface of the finger portion to the collection container.
3. The blood collection device according to any of the preceding clauses, further comprising a capillary tube that, in an assembled state, is arranged between the blood collection hole and the collection container, the capillary tube defining a fluid channel.
4. The blood collection device according clause 3, wherein an outer width of the capillary tube is larger than a width of the blood collection hole.
5. The blood collection device according to any of the preceding clauses, wherein a side wall of the finger portion comprises an air outlet hole.
6. The blood collection device according to any of the clauses 3-5, further comprising an insert member configured to be inserted into the finger portion and for receiving the capillary tube, to fixate the capillary tube with respect to the finger portion.
7. The blood collection device according to clause 6, wherein a capillary tube receiving channel of the insert member has a non-circular cross-section and wherein the capillary tube has a circular cross-section.
8. The blood collection device according to clause 6 or 7, wherein the finger portion, the insert member and the capillary tube are pre-assembled in the de-assembled state.
9. The blood collection device according to any of the preceding clauses, wherein an upper surface of the finger portion has an elongate shape
10. The blood collection device according to any of the preceding clauses, wherein a width of the base element is larger than 2.0 in.
11. The blood collection device according to any of the preceding clauses, wherein the base element comprises a receive pocket including the receive opening, for receiving the collection container in the assembled state of the blood collection device.
12. The blood collection device according to any of the preceding clauses, wherein the base element and/or the finger portion are manufactured via an injection moulding process, the base element and the finger portion preferable being made from polypropylene.
13. The blood collection device according to any of the preceding clauses, wherein the base element and/or the finger portion are manufactured via an 3D printing process, preferably via a selective laser sintering process, the base element and the finger portion preferable being made from PA2200.
14. A base element for a blood collection device, the base element having a width of more than 1.75 in and including an upstanding wall which defines a receive opening for receiving a collection container or a blood collection device, the upstanding wall having a height of less than 1.75 in,

wherein the base element is a separate part, configured to be shipped to a user in a de-assembled state with respect to the other parts of the blood collection device or with respect to the blood collection device, and

wherein the base element is configured to provide a stable support for the blood collection device in an assembled state.
15. Blood collection kit comprising the blood collection device according to any of the clauses 1-13 or a base element according to clause 14, a lancet, and a cap for sealing the collection container, preferably packed in a sterilized bag, the blood collection kit preferably also comprising a return envelope and/or an analysis tool for self-analysis of the collected blood sample.
16. Method for collecting a blood sample, comprising the steps of:

shipping a blood collection device according to any one of the clauses 1-13 or a blood collection kit according to clause 15 to a user by letterbox post, a base element of the blood collection device being de-assembled with respect to the other components of the blood collection device during shipment,

assembling the blood collection device;

pricking a finger of a patient to initiate a blood flow;

placing the finger of the patient on the finger portion of the blood collection device;

collecting the blood of the patient in the collection container;

de-assembling the blood collection device;

returning the collection container, inclusive of the blood collected therein;

wherein the steps of assembling the blood collection device, pricking the finger of a patient, placing the finger of the patient on the finger portion, collecting the blood, and de-assembling the blood collection device are all carried out by the patient him/herself.
17. A blood collection device for the self-collection of a blood sample by a user, the blood collection device comprising a base element, a collection container, and a finger portion,

the base element including an upstanding wall which defines a receive opening for receiving the collection container;

the collection container being configured for placement in the receive opening of the base element in a substantially upright orientation; and

the finger portion being configured for placement on top of the collection container and comprising a blood collection channel including a blood collection hole, wherein a width of the blood collection channel decreases in a direction from an upper surface of the finger portion to the collection container, and wherein the blood collection hole enables a flow of blood from the finger portion into the collection container.
18. The blood collection device according to clause 17, further comprising a capillary tube that, in an assembled state, is arranged between the blood collection hole and the collection container, the capillary tube defining a fluid channel.
19. The blood collection device according to clause 18, wherein an outer width of the capillary tube is larger than a width of the blood collection hole.
20. The blood collection device according to any of the clauses 17-19, wherein a side wall of the finger portion comprises an air outlet hole.
21. The blood collection device according to any of the clauses 18-20, further comprising an insert member configured to be inserted into the finger portion and for receiving the capillary tube, to fixate the capillary tube with respect to the finger portion.
22. The blood collection device according to clause 21, wherein a capillary tube receiving channel of the insert member has a non-circular cross-section and wherein the capillary tube has a circular cross-section.
23. The blood collection device according to any of the clauses 21-22, wherein at least the base element is a separate part, configured to be shipped to a user in a de-assembled state with respect to the other parts of the blood collection device, and

wherein the blood collection device is configured to be assembled by a user after receiving said blood collection device and before using said blood collection device.
24. The blood collection device according to at least the clauses 21 and 23, wherein the finger portion, the insert member and the capillary tube are pre-assembled in the de-assembled state.
25. The blood collection device according to any of the clauses 17-24, wherein an upper surface of the finger portion has an elongate shape
26. The blood collection device according to any of the clauses 17-25, wherein a width of the base element is larger than 1.75 in, preferably larger than 2.0 in and wherein a height of the of the upstanding wall is less than 1.75 in.
27. The blood collection device according to any of the clauses 17-26, wherein the base element comprises a receive pocket including the receive opening, for receiving the collection container in the assembled state of the blood collection device.
28. The blood collection device according to any of the clauses 17-27, wherein the base element and/or the finger portion are manufactured via an injection moulding process, the base element and the finger portion preferable being made from polypropylene.
29. The blood collection device according to any of the clauses 17-28, wherein the base element and/or the finger portion are manufactured via an 3D printing process, preferably via a selective laser sintering process, the base element and the finger portion preferable being made from PA2200.
30. Blood collection kit comprising the blood collection device according to any of the clauses 17-29, a lancet, and a cap for sealing the collection container, preferably packed in a sterilized bag, the blood collection kit preferably also comprising a return envelope and/or an analysis tool for self-analysis of the collected blood sample.
31. Method for collecting a blood sample, comprising the steps of:

shipping a blood collection device according to any one of the clauses 17-29 or a blood collection kit according to clause 30 to a user;

pricking a finger of a patient to initiate a blood flow;

placing the finger of the patient on the finger portion of the blood collection device;

collecting the blood of the patient in the collection container; returning the collection container, inclusive of the blood collected therein;

wherein the steps of pricking the finger of a patient, placing the finger of the patient on the finger portion, and collecting the blood are all carried out by the patient him/herself.

### LIST OF REFERENCE NUMERALS

1 Blood collection device

11 base element
W11 width of base element
H11 height of base element
111 upstanding wall
112 receive opening
113 receive pocket
114 hinge member

12 collection container
121 barline
122 outer tube
123 inner tube
124 outer rim of inner tube

13 finger portion
131 blood collection hole
W131 width blood collection hole
132 blood collection channel
W132 width blood collection channel
133 upper surface
134 side wall
135 air outlet hole

14 capillary tube
141 fluid channel

15 insert member
151 capillary tube receiving channel
100 blood collection kit

101 lancet

102 cap

103 sterilized bag

104 return envelope
200 finger

1. A blood collection device for the self-collection of a blood sample by a user, the blood collection device comprising a base element, a collection container, and a finger portion,
the base element having a width of more than 75 in and including an upstanding which defines a receive opening receiving the collection container. the upstanding wall having a height of less than 1.75 in;
the collection container being configured for placement in the receive opening of the base element in an upright orientation;
the finger portion being configured for placement on top of the collection container and comprising a blood collection channel including a blood collection hole, wherein a width the blood collection channel decreases in a direction from an upper surface of the finger portion to the collection container and wherein the blood collection hole, in an assembled state of the blood collection device, enables a flow of blood from the finger portion into the collection container;
wherein at least the base element is a separate part, configured to be shipped to a user in a de-assembled state with respect to the other parts of the blood collection device, and
wherein the blood collection device is configured to be assembled by a user after receiving said blood collection device and before using said blood collection device.

2. The blood collection device according to claim 1, further comprising a capillary tube that in an assembled state, is arranged between the blood collection hole and the collection container, the capillary tube defining a fluid channel.

3. The blood collection device according to claim 2, wherein an outer width of the capillary tube is larger than a width of the blood collection hole.

4. The blood collection device according to claim 1, wherein a side wall the finger portion comprises an air outlet hole.

5. The blood collection device according to claim 2, further comprising an insert member configured to be inserted into the finger portion and for receiving the capillary tube, to fixate the capillary tube with respect to the finger portion.

6. The blood collection device according to claim 5, wherein a capillary tube receiving channel of the insert member has a non-circular cross-section and wherein the capillary tube has a circular cross-section.

7. The blood collection device according to claim 5, wherein the finger portion, the insert member and the capillary tube are pre-assembled in the de-assembled state.

8. The blood collection device according to claim 1, wherein an upper surface of the finger portion an elongate shape.

9. The blood collection device according to claim 1, wherein a width of the base element is larger than 2.0 in.

10. The blood collection device according to claim 1, wherein the base element comprises a receive pocket including the receive opening, for receiving the collection container in the assembled state of the blood collection device.

11. The blood collection device according to claim 1, wherein the base element and the finger portion are manufactured via an injection moulding process.

12. The blood collection device according to claim 1, wherein the base element and the finger portion are manufactured via a 3D printing process.

13. Blood collection kit comprising the blood collection device according to claim 1, a lancet, and a cap for sealing the collection container.

14. (canceled)

15. The blood collection kit according to claim 13, wherein the blood collection kit is packed in a sterilized bag.

16. The blood collection kit according to claim 13, further comprising a return envelope.

17. The blood collection kit according to claim 13, further comprising an analysis tool for self-analysis of the collected blood sample.

18. Method for collecting a blood sample involving a blood collection device according to claim 1, comprising the steps of:
shipping the blood collection device to a user by letterbox post, a base element of the blood collection device being de-assembled with respect to the other components of the blood collection device during shipment,
assembling the blood collection device;
pricking a finger of a patient to initiate a blood flow;
placing the finger of the patient on the finger portion of the blood collection device;
collecting the blood of the patient in the collection container;
de-assembling the blood collection device;
returning the collection container, inclusive of the blood collected therein;
wherein the steps of assembling the blood collection device, pricking the finger of a patient, placing the finger of the patient on the finger portion, collecting the blood, and de-assembling the blood collection device are all carried out by the patient him/herself.

19. Method for collecting a blood sample involving a blood collection kit according to claim 13, comprising the steps of:
shipping the blood collection kit to a user by letterbox post, a base element of the blood collection device being de-assembled with respect to the other components of the blood collection device during shipment,
assembling the blood collection device;
pricking a finger of a patient to initiate a blood flow;
placing the finger of the patient on the finger portion of the blood collection device;
collecting the blood of the patient in the collection container;
de-assembling the blood collection device;
returning the collection container, inclusive of the blood collected therein;
wherein the steps of assembling the blood collection device, pricking the finger of a patient, placing the finger of the patient on the finger portion, collecting the blood, and de-assembling the blood collection device are all carried out by the patient him/herself.
